(19) United States
(12) Patent Application Publication
(10) Pub. No.: US 2014/0324062 A1
(43) Pub. Date: Oct. 30, 2014

(54) SCREWDRIVER FOR BONE SCREWS

(71) Applicant: Silony Medical International AG, Frauenfeld (CH)

(72) Inventors: Frank Heuer, Filderstadt (DE); Frank Thilo Trautwein, Filderstadt (DE); Michael Fromm, Mehrstetten (DE)

(73) Assignee: Silony Medical International AG, Frauenfeld (CH)

(21) Appl. No.: 14/263,603

(22) Filed: Apr. 28, 2014

(30) Foreign Application Priority Data
Apr. 29, 2013 (DE) 20 2013 004 369.5

(51) Int. Cl. A61B 17/88

(52) U.S. Cl. CPC A61B 17/8886
USPC 606/104

(57) ABSTRACT

A screwdriver for actuating bone screws which have a shaft and a fork head for holding a vertebral rot or the like, includes a central shaft, which at its distal end is provided with a formfitting or force-fitting profile for engagement in a corresponding counter profile on the bone screw for turning the bone screw, a distal sleeve which rotatably engages around the shaft and is provided with connecting means for holding the screwdriver on the fork head of the bone screw, a handle provided at the proximal end of the screwdriver and a turn handle which is connected with the distal sleeve, and an adjustment device for axial adjustment of the relative position of the central shaft relative to the distal sleeve in at least two different predefined positions which are adjusted for different sizes and shapes of bone screws.

### CROSS-REFERENCES TO RELATED APPLICATIONS

This application claims the priority of German Patent Application, Serial No. 20 2013 004 369.5, filed Apr. 29, 2013, pursuant to 35 U.S.C. 119(a)-(d), the disclosure of which is incorporated herein by reference.

### BACKGROUND OF THE INVENTION

The present invention relates to a screwdriver for actuating bone screws.

The following discussion of related art is provided to assist the reader in understanding the advantages of the invention, and is not to be construed as an admission that this related art is prior art to this invention.

Bone screws are screwed into a bone at a desired site, for example a vertebral bone in the body, and serve for fastening of for example a vertebral rod. The vertebral rod is fastened between multiple such screws at their fork head which is provided therefore. These bone screws usually have a threaded shaft and a fork head in the form of fork-like protruding legs, between which the vertebral rod is for example fastened via grub screws. The screw shaft is supported for rotation relative to the fork head and has at its rear end a formfitting profile or force-fitting profile in which a screwdriver can engage in order to screw the screw into the bone or out of the bone at the desired site. For this, conventional screwdrivers of this type are provided with a connecting or holding means, which holds the fork head of the screw during screwing in of the screw shaft. The bone screw is so to speak held at the fork head by the screwdriver so that an inner screw element enables the actuation of the bone screw.

Such bone crews are available with fork heads of different lengths to be able to use different fitting shapes of bone screws for example in the case of different relative positions of vertebral bones. This means in the case of so-called long-head bone screws that the legs of the fork head are longer relative to the so-called short-head bone screws. In order to enable the described actuation of such different types of bone screws, respective separate screwdrivers have been used in the state-of-the-art which where specifically adapted to the respective screw type. This has the disadvantage that on one hand multiple different screwdrivers have to be provided for the different types, versions and lengths of bone screws. On the other hand the handling of these different bone screws is made difficult because the user has to correctly associate the fitting screwdriver to the corresponding types of bone screws for example by a corresponding marking or coding.

It would therefore be desirable and advantageous to provide an improved to obviate prior art shortcomings and to provide a screwdriver for actuating bone screws, which can be used for different types, versions and lengths of bone screws. In addition the invention is intended to facilitate the correct use of fitting bones screw for the respective purpose of use.

### SUMMARY OF THE INVENTION

According to one aspect of the present invention, a screwdriver for actuating a bone screw with a shaft and a fork head for holding a vertebral rod or the like, the screwdriver includes a central shaft, having a distal end provided with a formfitting or force-fitting profile for engagement in a corresponding counter profile on the bone screw for turning off the bone screw; a distal sleeve rotatably overlapping the shaft and provided with connecting means for holding the screwdriver on the fork head of the bone screw; a handle at the proximal end of the screwdriver, a turn handle connected with the distal sleeve; and an adjustment device for axial adjustment of a position of the shaft relative to the distal sleeve in at least two different predefined positions which are adjusted for different sizes and shapes of the bone screws. The screwdriver according to the invention is thus variably adjustable to different perspective shapes or sizes of bone screws. With the adjustment device the relative position of the central shaft can be adjusted in a targeted manner with the formfitting or force fitting profile in relation to a sleeve, which engages around the shaft. Due to the at least two predefined different positions of the shaft relative to the sleeve for example so-called short-head bone screws as well as long-head bone screws can be actuated with the same screwdriver. In the respective different positions the adjustment device fixes the central shaft rotatably within the distal sleeve so that a holding of the bone screw with the connection means of the sleeve is enabled and at the same time the central shaft sufficiently protrudes so that the formfitting or force-fitting profile of the shaft sufficiently protrudes so as to allow actuation of the bone screw via turning of the screwdriver. The versatility of the screwdriver is thus enhanced. A changing between different screwdrivers in the case of different types of bone screws is thus no longer necessary.

According to an advantageous embodiment of the invention the adjustment device has a mechanical latching means for adjustment of different positions corresponding to different types or sizes of bone screws, which enables a predefined axial adjustment between the central shaft and a distal sleeve of the screwdriver. By means of a mechanical latching means a secure adjustment of the axial position of the shaft relative to the sleeve is ensured. The functional reliability of the mechanical latching means is high. By releasing the mechanical latching means the adjustment is enabled and after a latching, a secure fixing of the respective desired predefined position is given.

According to a further advantageous embodiment of the invention the adjustment means for adjusting the axial relative position is integrated in the turn handle of the distal sleeve. In this way the screwdriver remains compact in its outer shape and there are no additional upwardly protruding components by the adjustment device. In addition this facilitates the actuation of the adjustment device because the user can simply directly actuate on the turn handle for adjusting directly.

According to a further advantageous embodiment of the screwdriver according to the invention the central shaft, which is provided with a formfitting or force-fitting profile for turning the screw is provided with at least two concentric cross-sectional tapers into which a spring-loaded latching element of the adjustment device engages. The concentric cross-sectional tapers make it possible that the relative position and with this the position of the shaft relative to the distal sleeve can be accurately adjusted while still ensuring a turning of the shaft for actuating the bone screw. The adjustment device engages with corresponding counter shapes in the respective concentric cross-sectional tapers so that a secure fixing in the desired position is given.

According to a further advantageous embodiment of the invention, the adjustment device is configured to enable a certain axial adjustment or sliding of the relative position of the central shaft relative to the distal sleeve in each of the at least two predefined positions. With this, the distance of at least two positions relative to each other is exactly defined according to the desired length adjustment, while the shaft can still be lightly slid within this position relative to the sleeve for effecting the actuation. In this way in spite of the fixing by means of the adjustment device the inserting and retrieving of the formfitting or force-fitting profile into the bone screw is made possible. This facilitates the handling of the screwdriver.

According to a further advantageous embodiment of the invention the central shaft has a through opening. The central shaft is also provided with an inner opening, which traverses the entire length of the shaft. In this way a guiding wire or the like can be inserted into the screwdriver, which guiding wire extends up to the rear end of the screwdriver and can be used as positioning means during screwing in the bone screw. For example in this way a guiding wire which is fixed on the bone beforehand, can be guided through the screwdriver and the bone screw so that the bone screw can be screwed in at the correct position.

According to a further advantageous embodiment of the invention, the distal sleeve of the screwdriver itself is received in a rotatable outer sleeve. The rotatable outer sleeve prevents inadvertent loosening of the screw during fastening of the bone screw at its desired position. The outer sleeve also protects the distal sleeve situated there underneath and prevents thus a direct contact with the hand of the user.

According to a further advantageous embodiment of the invention, the shaft is spring-loaded in the direction towards the distal end of the screwdriver. With this the central shaft of the screwdriver is impinged with a slight pressure in the direction of the bone screw to be actuated. This preloading by the spring facilitates the handling of the screwdriver.

According to a further advantageous embodiment of the invention, the handle is releasably coupled with the central shaft at the proximal end of the screwdriver. In this way different shapes of shafts, for example with different formfitting profiles for different types of bone screws, can be used on the same handle. This also facilitates cleaning of individual parts of the screwdriver.

According to a further advantageous embodiment of the invention, the distance of the at least two predefined different positions of the arrangement of the shaft and the sleeve relative to each other correspond to a standardized difference in length of different fork heads of the bone screws. Thus standardized long-head bone screws and short-head bone screws can be actuated with the same screwdriver without problems.

According to a further advantageous embodiment of the screwdriver according to the invention, the adjustment device is a spring-loaded pushbutton which acts perpendicularly to the longitudinal direction of the screwdriver, which pushbutton has a recess which receives a shaft and a cutaway in the recess which cooperates with the cross-sectional tapers in such a manner that actuation of the pushbutton enables an adjustment of the shaft relative to the sleeve and an adjustment is prevented when the pushbutton is not actuated. With this the adjustment device is very compact. The spring-loaded pushbutton with the central recess for receiving the shaft also ensures a high functional reliability of the adjustment device.

According to a further advantageous embodiment of the invention, the distal sleeve has at its proximal end a thread which serves for fastening in a counter thread on the bone screw. At their fork head the bone screws usually have an inner thread, which serves for subsequent screwing in of a grub screw for fastening for example a vertebral rod. Into this thread the sleeve for holding the fork head can be screwed and after fastening the bone screw can be easily screwed out again.

According to a further advantageous embodiment of the screwdriver according to the invention, the handle is configured as a hand grip which extends transverse to the longitudinal direction of the shaft and is essentially a T-shaped hand grip on the proximal end of the screwdriver. This enables a simple operation of the screwdriver and a sufficient pressure can easily be generated by the user. In addition this also ensures that a user is able to securely hold the screwdriver with both hands.

According to a further advantageous embodiment of the invention, the turn handle is configured as a hollow tool handle, which extends in longitudinal direction of the shaft. In the interior of the hollow space of the turn handle the adjustment device according to the invention can thus easily be integrated. This also simplifies the handling of the screwdriver, because during screwing the bone screws in or out the user can securely hold the screwdriver on the turn handle with one hand and with the other hand on the front handle.

According to a further advantageous embodiment of the invention, a guiding wire is provided in the interior of the through opening of the shaft, which guiding wire serves for defining and for locating the correct position of the desired site of the bone screw in the bone. The guiding wire can completely be guided through the interior of the shaft without interfering with the function of the screwdriver and after placing the bone screw can be easily removed again. Such a guiding wire is usually fastened to the bone beforehand so that the bone screw, which is threaded thereon can be screwed in at the right site at the bone.

According to a further advantageous embodiment of the invention, the formfitting or force-fitting profile of the screwdriver and/or the connecting means is/are configured replaceable on the distal sleeve. The versatility of the screwdriver is thus increased. Different shapes, sizes or types of bone screws can be actuated with the same screwdriver by exchanging the corresponding parts

### BRIEF DESCRIPTION OF THE DRAWING

Other features and advantages of the present invention will be more readily apparent upon reading the following description of currently preferred exemplified embodiments of the invention with reference to the accompanying drawing, in which:

FIG. 1a is a side view of an exemplary embodiment of a screwdriver according to the invention in an operating position for use in bone screws with short fork head;

FIG. 1b is a side view of the exemplary embodiment shown in FIG. 1a of a screwdriver according to the invention in an operating position for use in bone screws with long for head;

FIG. 2 is a perspective view of the exemplary embodiment of a screwdriver according to the invention;

FIG. 3 is a perspective exploded view of the exemplary embodiment of a screwdriver according to the invention;

FIG. 4a is a side view of the exemplary embodiment of the screwdriver according to the invention;

FIG. 4b is a longitudinal sectional view of the exemplary embodiment of the screwdriver according to the invention taken along the line A-A of FIG. 4a;

FIG. 4c is a further side view of the exemplary embodiment of a screwdriver according to the invention; and

FIG. 4d is a cross-sectional view of the exemplary embodiment of the screwdriver according to the invention taken along the line B-B of FIG. 4c.

### DETAILED DESCRIPTION OF PREFERRED EMBODIMENTS

Throughout all the Figures, same or corresponding elements are generally indicated by same reference numerals. These depicted embodiments are to be understood as illustrative of the invention and not as limiting in any way. It should also be understood that the drawings are not necessarily to scale and that the embodiments are sometimes illustrated by graphic symbols, phantom lines, diagrammatic representations and fragmentary views. In certain instances, details which are not necessary for an understanding of the present invention or which render other details difficult to perceive may have been omitted.

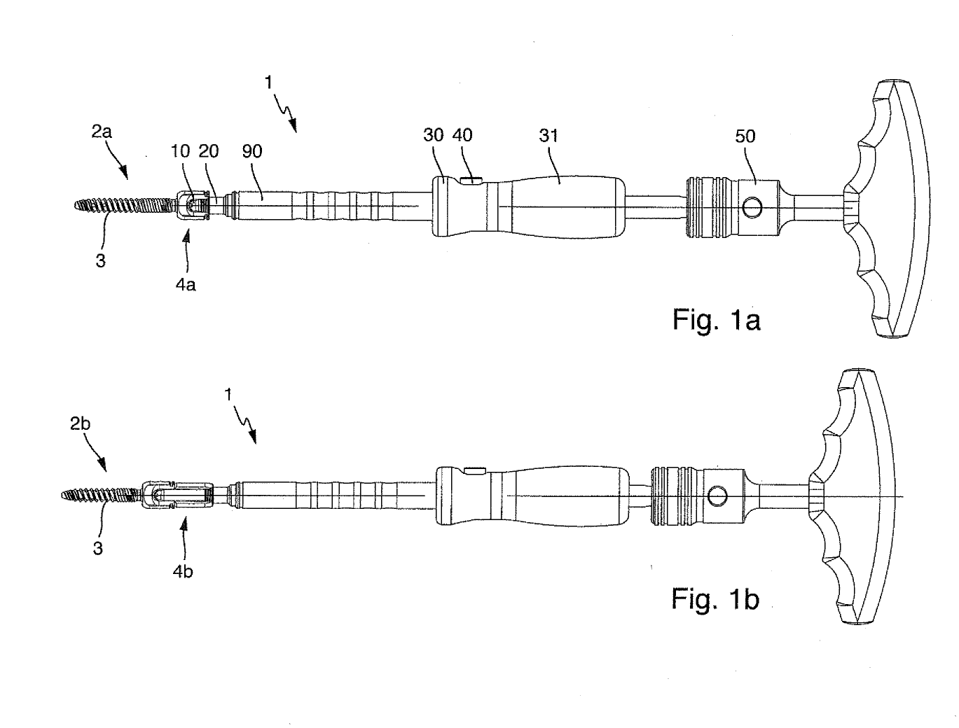

Turning now to the drawing, and in particular to FIGS. 1a and 1b there is shown in respective side views an exemplary embodiment of a screwdriver 1 according to the invention for bone screws 2a, 2b. FIG. 1 shows the exemplary embodiment of the screwdriver 1 according to the invention in an operating position for use in bone screws 2a with a short fork head 4a, i.e., a fork head whose laterally protruding legs are configured relatively short. FIG. 1 b shows the screwdriver 1 in an operating position for use in a different type of bone screws 2b, i.e. bone screws 2b, which have a long fork head 4b. The bone screws 2a, 2b have a shaft 3 and a fork head 4a, 4b rotatably connected with the shaft. The front end of the screwdriver 1 is provided with a formfitting profile on the shaft 10, for example a Torx-profile, which serves for turning the screw shaft 3. In order to be able to actuate different types of bone screws 2a, 2b with the same screwdriver 1 according to the invention, the screwdriver 1 is provided with an adjustment device 40 for adjusting the relative position between the central shaft 10 and an outer sleeve 20. The adjustment device 40 is here realized in the form of a pushbutton in a turn handle 30 whose actuation allows adjustment of the central shaft from its position shown in FIG. 1a for short bone screws 2a into the position of FIG. 1b which is adjusted for bone screws 2b with a longer fork head which position is extended relative to the first position. At its proximal end the screwdriver 1 has a handle 50, which serves for enabling a user to hold the screwdriver with one hand, while with the other hand holding the screwdriver on the turn handle 30. The inner shaft 10 can be turned via the handle 50 while the distal sleeve 20 is fastened on the fork head 4a, 4b via the thread (see FIG. 1a and FIG. 1b). On the outside of the distal sleeve 20 a turnable outer sleeve 90 is provided so as to prevent inadvertent loosening. In the following the special shape and configuration of the adjustment device 40 is explained in more detail with reference to the further figures of this embodiment.

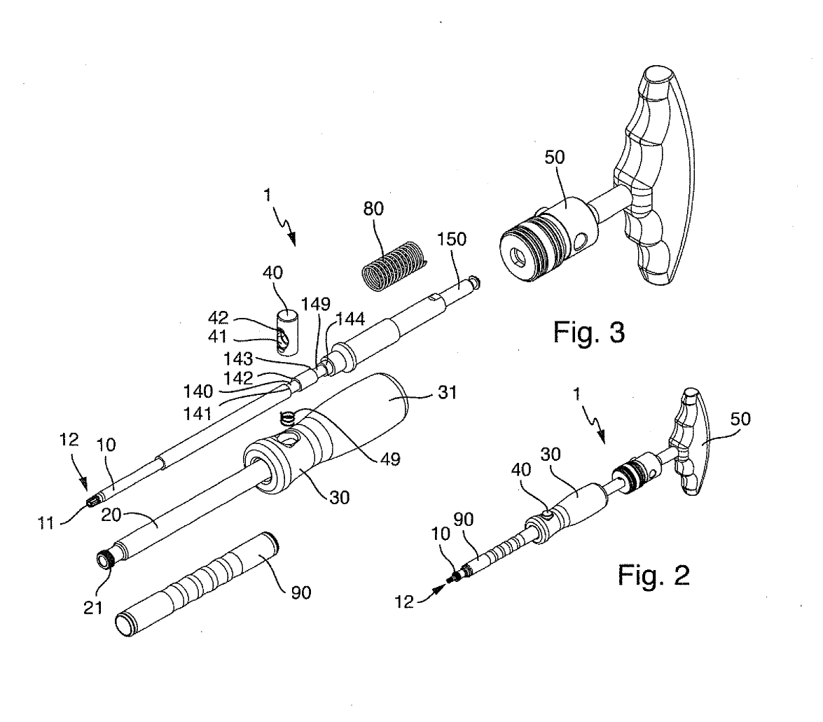

The exemplary embodiment of the screwdriver according to the invention is shown in FIG. 2 in a perspective view and in FIG. 3 in a perspective exploded view. The screwdriver 1 for bone screws according to the invention has in this exemplary embodiment a central shaft 10, which at its distal end has a formfitting profile 12 which serves for engagement of a corresponding counter profile on the bone screw. The formfitting profile 12 can for example be a Torx-contour or another profile present in these type of bone screws which serves for transmitting a torque to the bone screw. The proximal end the central shaft 10 is provided with a connecting pin 150, which serves for releasable fastening on the handle 50. The shaft 120 is rotatably received in the interior of a distal sleeve 20 and a turn handle 30. At its free end the sleeve 20 is provided with a thread 21 for connection with a corresponding internal thread on the fork head of the bone screw to be actuated (see FIG. 1a and FIG. 1b). In the turn handle 30 an adjustment device 40 according to the invention in the form of a pushbutton is realized, by means of which an axial position between the shaft 10 and the sleeve 20 can be adjusted. The adjustment device 40 is composed of a spring-loaded pushbutton with a recess 42 through which the shaft 10 is guided in the assembled state. In the recess 42 a cutaway 41 is provided which interacts with two concentric cross sectional tapers 140, 149 of the shaft 10 in order to set the predefined positions during adjustment with the adjustment device 40. When actuating the pushbutton 40, the shaft 10 can be adjusted from one of the concentric cross sectional tapers 140 to the other concentric cross sectional taper 149 and vice versa. In this way the different adjustments of the screwdriver 1 are on one hand enabled for the bone screws 2a with short fork head 4a and on the other hand for the bone screws 2b with long fork head 4b which are shown in the above FIGS. 1a and 1b.

The distal sleeve 20 is received in a rotatable outer sleeve 90 so that inadvertent loosening of the bone screw is avoided when the bone screw is turned with the screwdriver 1. In this exemplary embodiment the shaft 10, which serves for screwing the bone screws, is pre-tensioned with a pressure spring 80, so as to enable easier operation of the screwdriver 1. For this, the shaft 10 is provided with a collar-like protrusion against which the pressure spring 80 comes to rest in the interior of the turn handle 30 so that the shaft 10 is always pre-tensioned in the direction toward the bone screw to be actuated.

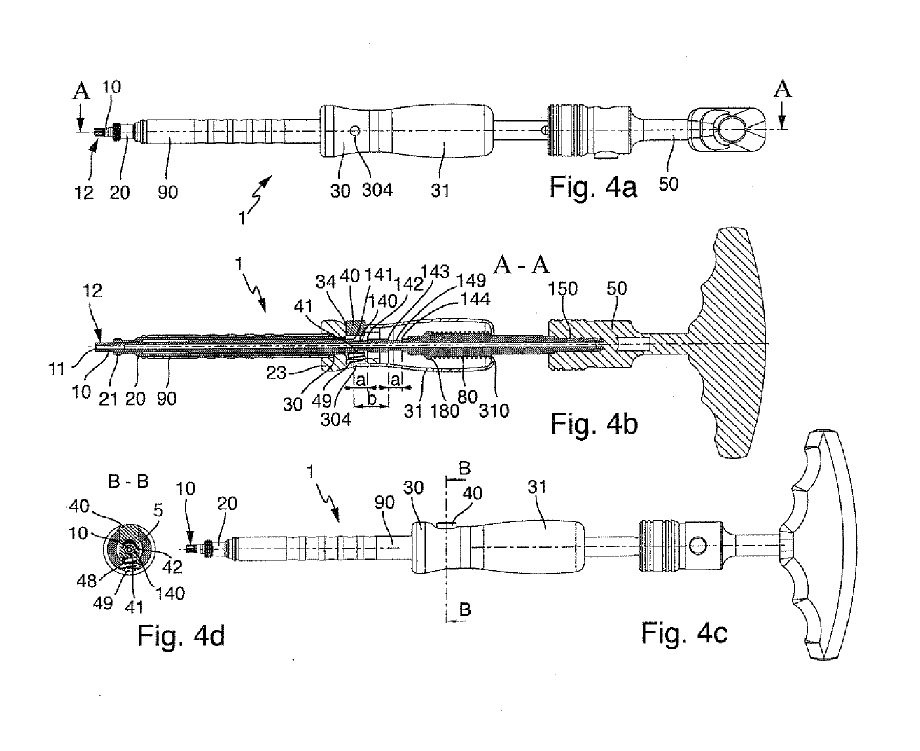

FIGS. 4a, 4b, 4c and 4d show respective side views or sectional views of the exemplary embodiment of a screwdriver 1 according to the invention by way of which the adjustment device 40 is described in the following in more detail. As can be seen in particular from the longitudinal sectional view of FIG. 4b the screwdriver 1 has a central shaft 10, which is supported in the interior of a distal sleeve 20 and a turn handle 30. The shaft 10 is connected for co-rotation with the handle 50 via a connecting pin 150, so that the handle 50 can be removed from the shaft if needed. The shaft 10 has at its distal end a form fitting profile 12, which can for example be a Torx-contour or another profile appropriate for actuating screws. On the front end the shaft 10 is rotatably received in a distal sleeve 20, which is fixedly connected with the turn handle 30. The distal sleeve 20 has a thread 21 which serves for fastening the holder of the bone screw when turning the screw. The distal sleeve 20 is received in a recess 90 so that an inadvertent loosening of the screw during turning is prevented. The turn handle 30 is configured hollow, so that on one hand the shaft 10 can be rotatably guided in its interior and on the other hand the adjustment device 40 according to the invention can be received in the turn handle 30.

In this exemplary embodiment the adjustment device of the screwdriver 1 is provided in the form of a spring-loaded pushbutton 40, which is integrated in the turn handle 30. The pushbutton 40 is essentially inserted transverse to the longitudinal direction of the screwdriver 1 in an opening in the turn handle 30 provided therefor. The pushbutton 40 is provided with a recess 42 through which the shaft 10 is guided. In the recess 42 of the pushbutton 40 an cutaway 41 is present which can be seen in particular when comparing the longitudinal sectional view of FIG. 4b and the cross sectional view of FIG. 4d. The cutaway 41 is adjusted to the shape of two concentric cross-sectional tapers 140, 149 of the shaft 10. When the pushbutton 40 is not actuated (position of FIGS. 4b and 4d) the cross-sectional taper 140 is in engagement with the cutaway 41 so that the relative axial position between the shaft 10 and the sleeve 20 is fixed. When the pushbutton 40 is actuated against the force of the spring 49, the shaft 10 can be adjusted in the axial direction. The adjustment travel between the two cross-sectional tapers 140, 149 is designated “b” In FIG. 4b. This adjustment travel “b” corresponds to a difference in length of the fork head 4a, 4b of different sized bone screws 2a, 2b as they are shown in FIGS. 1a and 1b. Each of the cross-sectional tapers 140, 149 is provided with rear and front delimiting surfaces 141, 142, 143, 144 against which the cutaway 41 of the pushbutton 40 abuts. The shaft 10 can thus be somewhat adjusted relative to the sleeve 20 or the turn handle 30 within the respective predefined position, which is indicated with the distances “a” in FIG. 4b. In this way a certain displacement within the delimiting surfaces 141, 142 or 143, 144 is possible while the adjustment device 40 still maintains the selected predefined position. In the interior of the turn handle 38 a pressures spring 80 is received, which rests against a support surface 180 and a rear wall 310 of the turn handle so that the shaft 10 is pre-tensioned with a slight pressure in the direction of the distal end of the screwdriver 1. This pre-tension facilitates operation of the screwdriver 1. At the height of the pushbutton 40 the turn handle 30 is provided on an opposing side with an opening 304, which facilitates cleaning of the interiorly situated components.

As can be seen in particular in the cross-sectional view of FIG. 4d, the pushbutton of the adjustment device 40 in this exemplary embodiment is provided with a central approximately circular-shaped recess 42, which enables passage and displacement of the shaft 10 also outside of the cross-sectional taper 140. The pushbutton 40 has a hollow space 48 in which a pressure spring 49 is received by which the pushbutton 40 is pre-tensioned. In the pre-tensioned position the cross-sectional taper 140 of the shaft 10 is positioned in the cutaway 41 which is adjusted to the small diameter of the cross-sectional taper 140 so that the shaft 10 is securely fixed in its axial position.

With the screwdriver according to the invention, different sizes or types of bone screws 2a, 2b can be turned with the same screwdriver. By simple adjustment of the adjustment device 40 from a predefined position into another predefined position the different types or sizes of bone screws can be actuated. The screwdriver according to the invention is constructed relatively simple and has a compact design. The mechanical adjustment device 40 is completely integrated in the interior of the turn handle. Providing the possibility of adjustment by means of the adjustment device 40 increases the possible applications and the variability of the screwdriver 1.

The present invention is not limited to the shown embodiments and different changes and modifications can be made within the scope of the included claims: for example more than two predefined axial positions of the shaft 10 relative to the turn handle 30 or the sleeve 20 can be provided so that an even greater number of different types of bone screws can be turned with the same screwdriver 1.

In addition instead of the described mechanical adjustment device 40 and electric or otherwise configured adjustment device can be provided. The adjustment device is also not limited to a spring-loaded pushbutton and other means for the axial adjustment of the shaft in the different positions can be provided as it is known to a person skilled in the art. Also the screwdriver according to the invention is not limited to the configuration of the handle 50 and the turn handle 30. Instead of an essentially T-shaped a handle 50 a conventional axial screwdriver handle can for example be provided. The connection of the handle 50 to the shaft 10 can be configured releasable, however, a fixed connection between the shaft 10 and the handle 50 can also be provided.

While the invention has been illustrated and described in connection with currently preferred embodiments shown and described in detail, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and includes equivalents of the elements recited therein:

1. A screwdriver for actuating a bone screw with a shaft and a fork head for holding a vertebral rod or the like, the screwdriver comprising:
a central shaft, having a distal end provided with a formfitting or force-fitting profile for engagement in a corresponding counter profile on the bone screw for turning of the shaft of the bone screw;
a distal sleeve rotatably overlapping the shaft and provided with connecting means for holding the screwdriver on the fork head of the bone screw;
a handle provided at a proximal end of the screwdriver;
a turn handle connected with the distal sleeve; and
an adjustment device for axial adjustment of a position of the shaft relative to the distal sleeve in at least two different predefined positions which are adjusted for different sizes and shapes of the bone screws.

2. The screwdriver of claim 1, wherein the adjustment device comprises a mechanical latching means, which enables a predefined axial adjustment between the shaft and the distal sleeve.

3. The screwdriver of claim 1, wherein the adjustment device is integrated in the turn handle of the distal sleeve.

4. The screwdriver of claim 1, wherein the central shaft is provided with at least two concentric cross-sectional tapers, and wherein the adjustment device comprises a spring-loaded latching element engaging in the at least two concentric cross-sectional tapers.

5. The screwdriver of claim 1, wherein the adjustment device is configured to allow a defined axial adjustment of the position of the central shaft relative to the distal sleeve in each of the at least two predefined positions.

6. The screwdriver of claim 1, wherein the central shaft has a through opening.

7. The screwdriver of claim 1, further comprising a rotatable outer sleeve, the distal sleeve being received in the rotatable outer sleeve.

8. The screwdriver of claim 1, wherein the central shaft is spring-loaded in a direction towards a distal end of the central shaft.

9. The screwdriver of claim 1, wherein the handle is releasably coupled with the central shaft.

10. The screwdriver of claim 1, wherein a distance between the central shaft and the sleeve in the at least two predefined different positions corresponds to a standardized length difference of fork heads of the bone screws.

11. The screwdriver of claim 4, wherein the adjustment device is constructed as a pushbutton which acts perpendicularly to the longitudinal direction of the screwdriver and which has a recess which receives the shaft, said recess having a cutaway provided therein, said cutaway interacting with the concentric cross-sectional tapers such that when actuating the pushbutton adjustment of the central shaft to the distal sleeve is enabled and when not actuating the pushbutton is adjustment of the central shaft to the distal sleeve is disabled.

12. The screwdriver of claim 1, wherein the distal sleeve has a proximal end provided with a thread for fastening in a counter thread of bone screw.

13. The screwdriver of claim 1, wherein the handle is a T-shaped handle provided on the proximal end of the screwdriver and extending transverse to a longitudinal direction of the central shaft.

14. The screwdriver of claim 1, wherein the turn handle is configured as a tool handle which extends in a longitudinal direction of the central shaft and has an inner hollow space.

15. The screwdriver of claim 1, further comprising a guiding wire provided in an interior of the through opening of the shaft, for defining and locating a position of a site of the bone at which the bone screw is to be screwed into the bone.

16. The screwdriver of claim 1, wherein the formfitting or force-fitting profile of the screwdriver and/or the connecting means on the distal sleeve is configured exchangeable.
